(19) United States
(12) Patent Application Publication
(10) Pub. No.: US 2018/0296769 A1
(43) Pub. Date: Oct. 18, 2018

(54) SAFETY SYRINGE FOR INJECTING REGULAR INSULIN

(71) Applicants: Hsien-Tsung WANG, Taipei (TW); ANHUI HONGYU WUZHOU MEDICAL MANUFACTURER CO., LTD., Beijing (CN)

(72) Inventors: HSIEN-TSUNG WANG, Taipei (TW); HONG-YU ZHANG, Beijing (CN)

(21) Appl. No.: 15/489,976

(22) Filed: Apr. 18, 2017

(51) Int. Cl. A61M 5/32; A61M 5/34

(52) U.S. Cl. CPC A61M 5/3221; A61M 5/3202; A61M 2202/04; A61M 5/343; A61M 2005/3223; A61M 5/3276

(57) ABSTRACT

A safety syringe (1) includes a sleeve assembly (10) threadedly secured to a vial (40) containing regular insulin and including an axial channel (12) with a needle assembly (20) placed therein, and two opposite pivot pins (14) formed on an outer surface. The needle assembly (20) includes a needle hub (22) formed on a front end, and a hypodermic needle (23) through the needle hub (22). A protective covering (30) includes two opposite arms (31) pivotably secured to the pivot pins (14), a space (32), a slot (33) parallel to the arms (31) and communicating with the space (32), and at least one lock member (34) at an end of the slot (33). After injection, a clockwise rotation of the protective covering (30) until the lock member (34) passes through the needle hub (22), and the hypodermic needle (23) passes through the slot (33) to conceal in the space (32).

### BACKGROUND OF THE INVENTION

### 1. Field of the Invention

The invention relates to safety syringes and more particularly to a safety syringe for injecting regular insulin, the hypodermic needle of the safety syringe being permanently locked in a concealed state so as to prevent it from pricking individuals and being reused.

### 2. Description of Related Art

Rates of type 2 diabetes have increased markedly since 1960. As of 2013 there were approximately 368 million people diagnosed with the disease. Typically, diabetes begins in middle or older age and it is a long term metabolic disorder that is characterized by high blood sugar, insulin resistance, and relative lack of insulin. Common symptoms include increased thirst, frequent urination and unexplained weight loss. Symptoms may also include increased hunger, feeling tired, and sores that do not heal. Often symptoms come on slowly. Long-term complications from high blood sugar include heart disease, strokes, diabetic retinopathy which can result in blindness, kidney failure, and poor blood flow in the limbs which may lead to amputations. One of the anti-diabetic medications is regular insulin injection.

A conventional syringe for injecting regular insulin includes a needle assembly for piercing a vial and drawing medicine therefrom, and a protective sleeve placed on the needle assembly after injecting the medicine into the skin of a patient. The used syringe is discarded. It is strongly suggested that handle of the used syringe should be very careful in order to prevent contamination. Further, the unprotected hypodermic of the used syringe may prick a medical employee if sufficient care is not taken. Further, reuse of the used syringe is still possible because the protective sleeve is not well fastened after the injection. Thus, the need for improvement still exists.

### SUMMARY OF THE INVENTION

It is therefore one object of the invention to provide a safety syringe for injecting regular insulin, the hypodermic needle of the safety syringe being permanently locked in a concealed state so as to prevent it from pricking individuals and being reused.

For achieving above and other objects, the invention provides a safety syringe for injecting regular insulin comprising a sleeve assembly; a needle assembly; and a protective covering; wherein the sleeve assembly is threadedly secured to a via containing regular insulin and includes an axial channel with the needle assembly placed therein, and two opposite pivot pins formed on an outer surface; the needle assembly includes a needle hub formed on a front end, and a hypodermic needle disposed through the needle hub; the protective covering includes two opposite arms pivotably secured to the pivot pins, a space, a slot parallel to the arms and communicating with the space, and at least one lock member at an end of the slot; and after injection, a clockwise rotation of the protective covering until the lock member passes through the needle hub, and the hypodermic needle passes through the slot to conceal in the space.

According to an embodiment of the present invention, the at least one lock member is inclined.

According to an embodiment of the present invention, the at least one lock member is inclined toward the space.

According to an embodiment of the present invention, the number of the at least one lock member is two and the lock members are opposite.

According to an embodiment of the present invention, the number of the at least one lock member is two and the lock members are offset.

According to an embodiment of the present invention, the sleeve assembly further comprises two sets of upper and lower limit members on an outer surface.

According to an embodiment of the present invention, the protective covering further comprises a gripping member on a rear surface.

According to an embodiment of the present invention, further comprising a cap placed on the sleeve, the cap including a rear enlargement, a forward member, and two left and right sides spaced cuts and one lower cut on the enlargement.

The second aspect of the present invention is provides a sleeve assembly; a needle assembly; and a protective sleeve; wherein: the sleeve assembly is threadedly secured to a via containing regular insulin and includes an axial channel with the needle assembly placed therein; the needle assembly includes a hollow cylinder, a needle hub formed on a front end of the hollow cylinder and having a protuberance formed on an outer surface, and a hypodermic needle disposed through the needle hub; the protective sleeve includes a lengthwise guide groove formed on an inner surface with the protuberance disposed therein after placing the protective sleeve on the needle hub, a lengthwise positioning groove formed on the inner surface and parallel to the guide groove, the positioning groove having a riser and a well; and after injection, a rotation of the protective sleeve and a pulling of the protective sleeve until the protuberance passes the riser to enter the well to be permanently locked therein.

According to an embodiment of the present invention, the riser is inclined toward a forward end.

According to an embodiment of the present invention, the sleeve assembly further comprises a first groove formed on the inner surface and being open to a front end, a second groove formed on the inner surface and being parallel to the first groove, the second groove having a projection and a cavity adjacent to the projection, and a third groove interconnecting a rear end of the first groove and a rear end of the second groove; the hollow cylinder includes a protrusion formed on an outer surface; and the protrusion is complimentarily disposed in the first groove in a prior to use position.

According to an embodiment of the present invention, the projection is inclined toward a rear end.

The above and other objects, features and advantages of the invention will become apparent from the following detailed description taken with the accompanying drawings.

### BRIEF DESCRIPTION OF THE DRAWINGS

FIG. 1 is a perspective view of a safety syringe for injecting regular insulin according to a first preferred embodiment of the invention with a cap;

FIG. 2 is a perspective, exploded view of the safety syringe with the needle protector removed;

FIG. 3 is an exploded view of the safety syringe;

FIG. 4 is a perspective view of FIG. 2 is a perspective, exploded view of the safety syringe with the needle protector and the cap removed and a vial secured thereto;

FIG. 5 is a longitudinal sectional view of the front portion of FIG. 4;

FIG. 6 is a view similar to FIG. 4 showing a covering of the hypodermic needle after use;

FIG. 7 is a longitudinal sectional view of the front portion of FIG. 6;

FIG. 8 is a perspective view of a safety syringe for injecting regular insulin according to a second preferred embodiment of the invention;

FIG. 9 is an exploded view of the safety syringe of FIG. 8;

FIG. 10A is a perspective view of the sleeve assembly;

FIG. 10B is a sectional view of the protective sleeve;

FIG. 11 is a perspective view of the safety syringe of FIG. 8 with the needle protector and the cap removed and a vial secured thereto;

FIG. 12 is a longitudinal sectional view of the front portion of FIG. 11;

FIG. 13 is an exploded perspective view showing a removal of the vial and a covering of the hypodermic needle after use; and

FIG. 14 is a longitudinal sectional view of the front portion of FIG. 13.

### DETAILED DESCRIPTION OF THE INVENTION

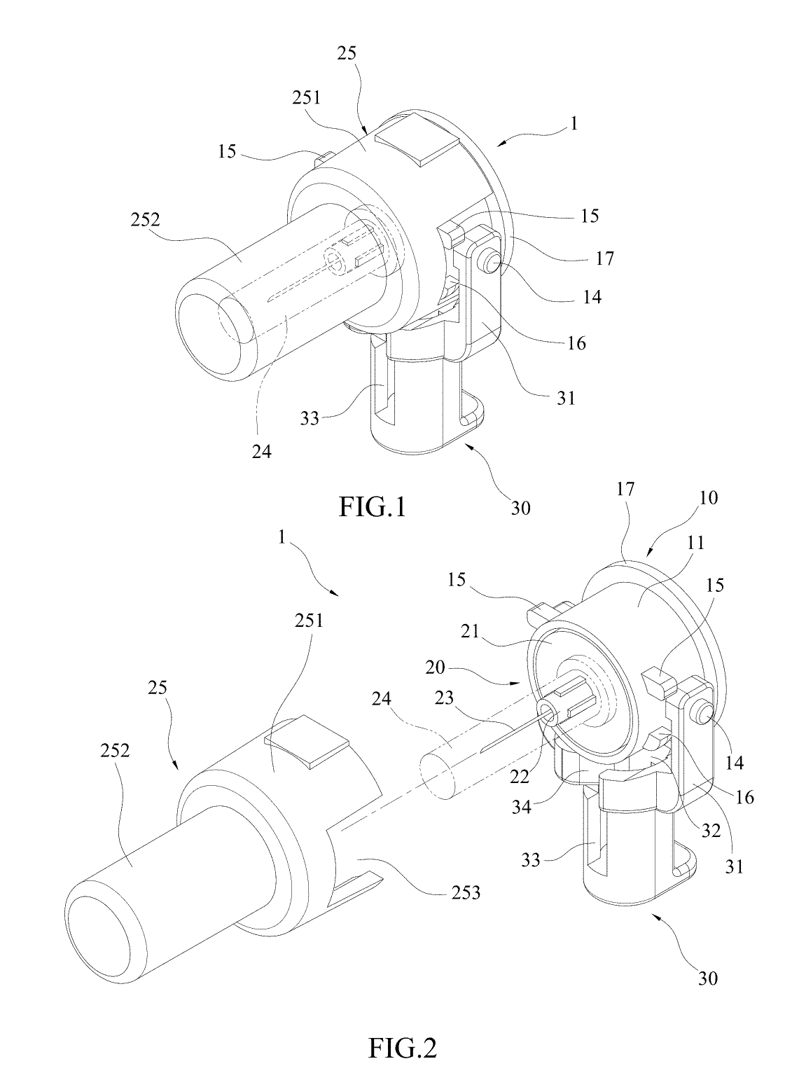

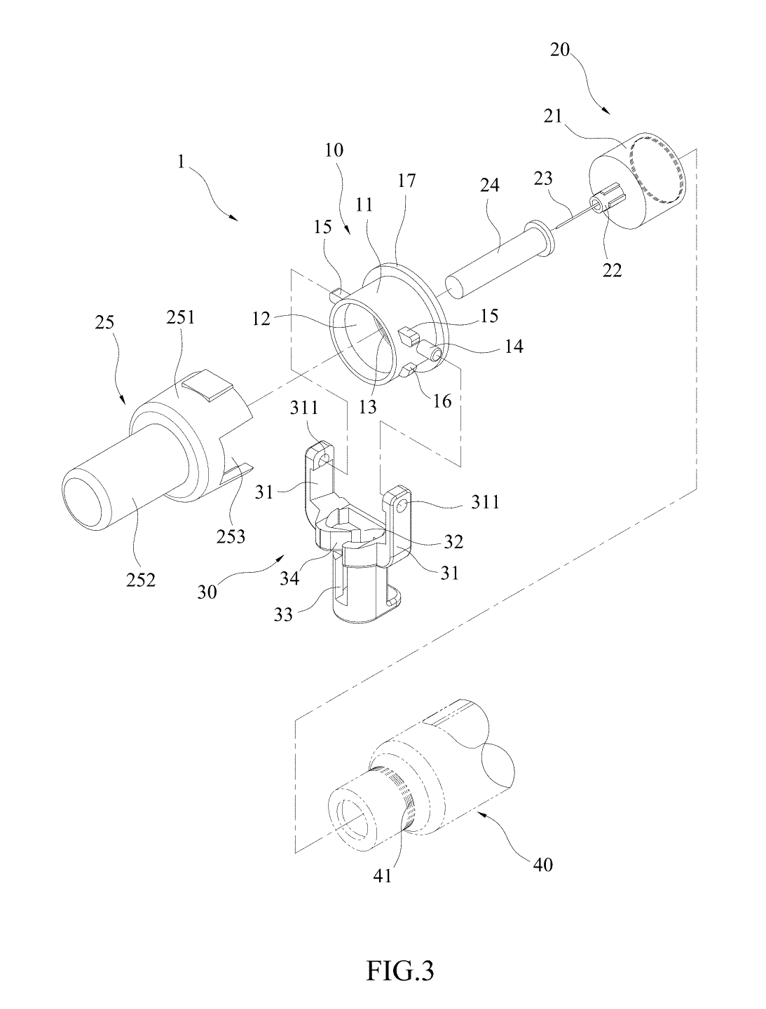

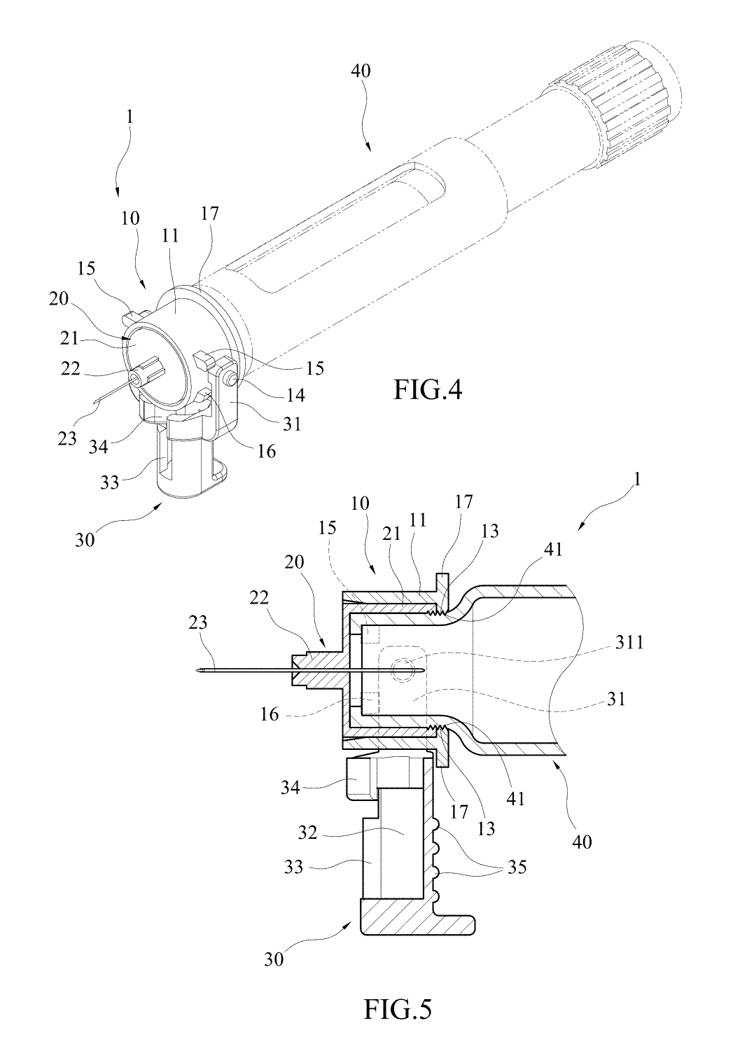

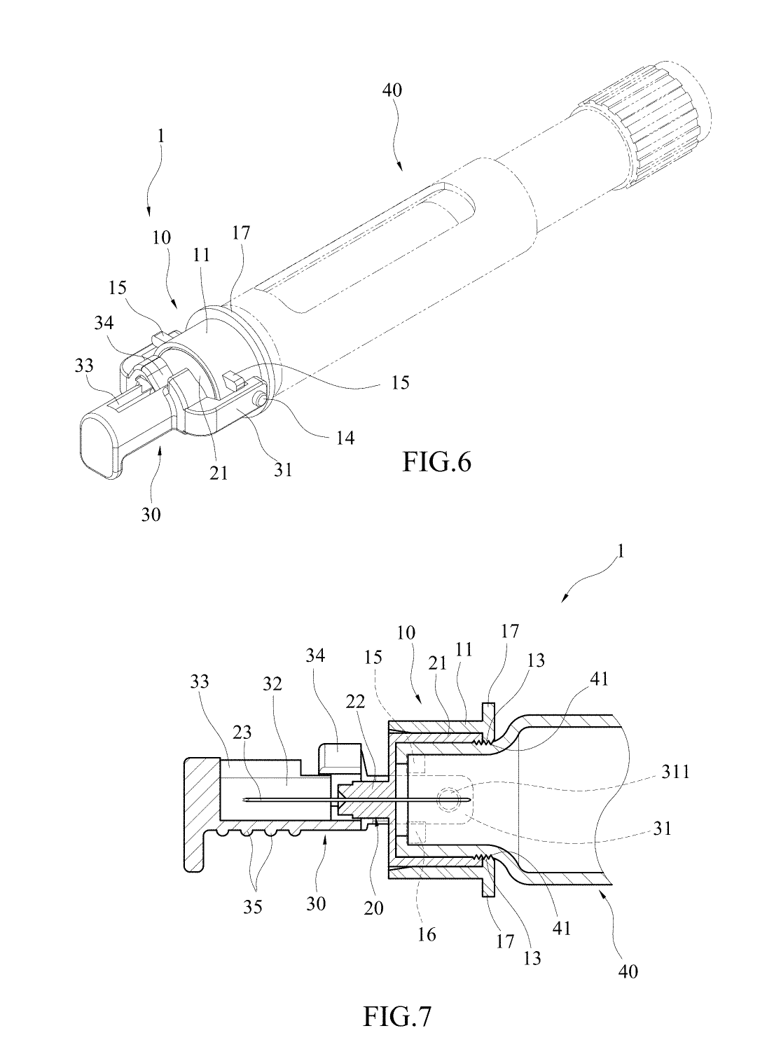

Referring to FIGS. 1 to 7, a safety syringe 1 for injecting regular insulin in accordance with a first preferred embodiment of the invention comprises the following components as discussed in detail below.

A sleeve assembly 10, a needle assembly 20, and a protective covering 30 are provided. The sleeve assembly 10 includes a sleeve 11, an axial channel 12 through the sleeve 11 with the needle assembly 20 mounted therein, a rear annular threaded rim 13 on an inner surface of the sleeve 11, two opposite pivot pins 14 on an outer surface of the sleeve 11, two sets of triangular upper and lower limit members 15 and 16 on the outer surface of the sleeve 11, and a rear flange 17.

The needle assembly 20 includes a hollow cylinder 21 with the sleeve 11 placed thereon, a needle hub 22 formed on a front end of the cylinder 21, a hypodermic needle 23 disposed through the needle hub 22, a needle protector 24 releasably placed on both the needle hub 22 and the hypodermic needle 23 prior to use, and a cap 25 placed on the sleeve 11 and engaged the flange 17, the cap 25 including a rear enlargement 251, a forward member 252, and relative to both sides of the sleeve 11 on the upper and lower limit members position cut a left and right spaced cuts 253, 253, and cut one notch (not marked) on the lower position of the enlargement 251 to put on the sleeve assembly 10.

The protective covering 30 includes two opposite arms 31 in the cuts 253 respectively, the arm 31 having a pivot hole 311 with the pivot pin 14 pivotably secured thereto, a space 32, and a knurled member 35 on a rear surface of the protective covering 30. The lock members 34 may be offset in other embodiments.

A vial 40 contains regular insulin and includes front external threads 41 configured to threadedly secure to the annular threaded rim 13 to fasten the vial 40 and the sleeve assembly 10 together. In an assembled state and prior to use, the protective covering 30 is disposed directly under the sleeve assembly 10, the vial 40 is secured to the rear end of the sleeve assembly 10, and thus the needle assembly 20, the sleeve assembly 10, and the vial 40 aligned.

A medical employee may remove the cap 25 and the needle protector 24 prior to injecting regular insulin in the vial 40 into the skin of a patient through the hypodermic needle 23. After use, the medical employee may clockwise pivot the protective covering 30 about 90 degrees until the lock members 34 pass through the needle hub 22, the hypodermic needle 23 passes through the slot 33 to conceal in the space 32, and the arms 31 are stopped by the upper limit members 15. Thereafter, the medical employee may detach the empty vial 40 from the sleeve assembly 10 prior to properly disposing the used safety syringe 1.

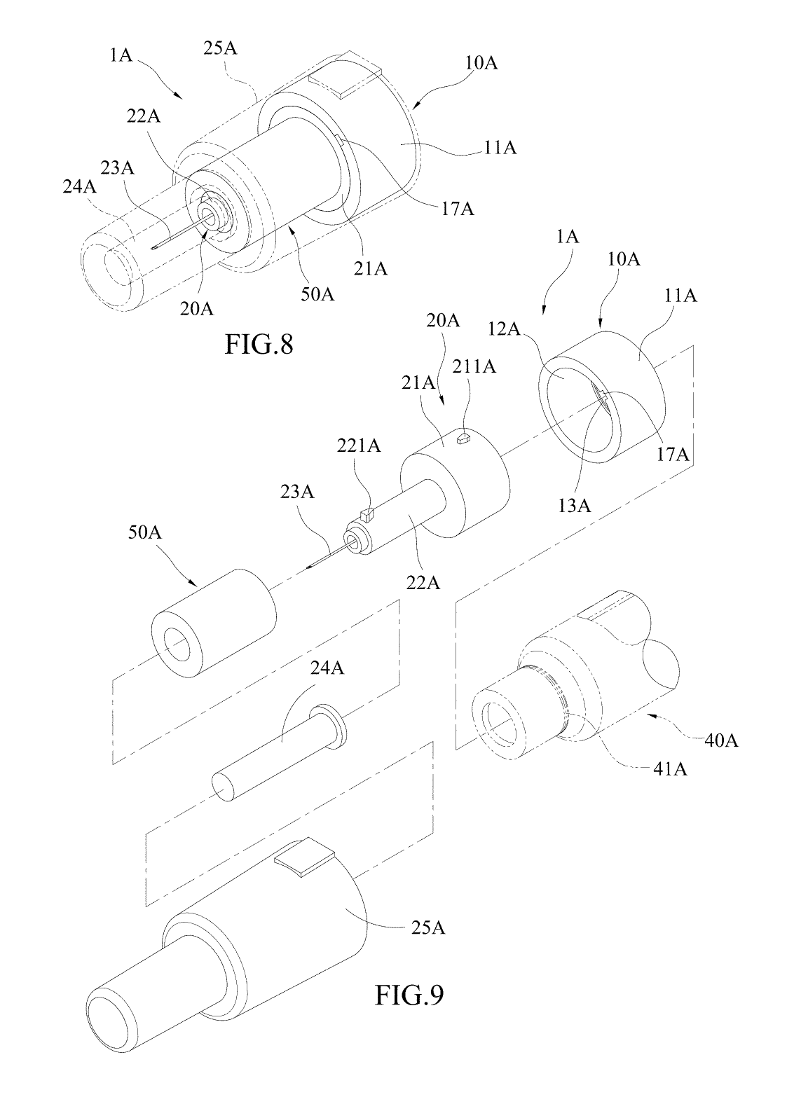

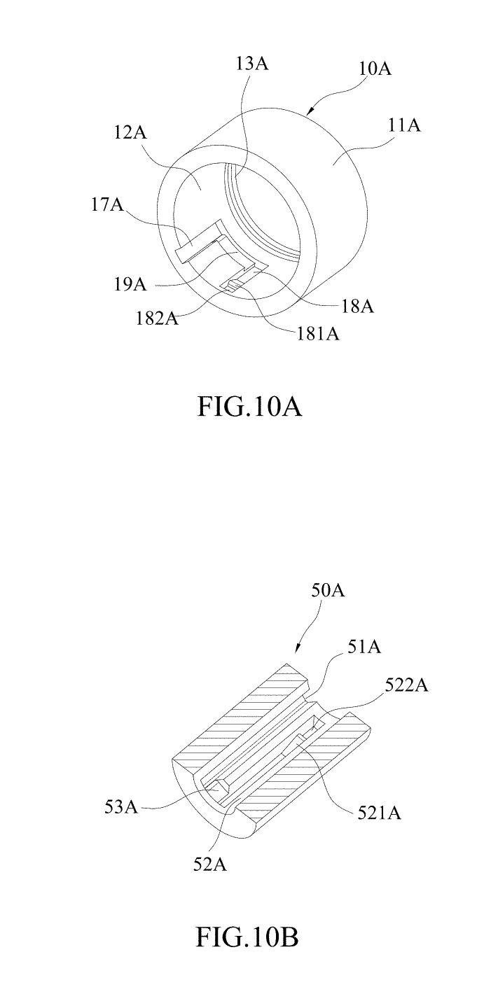

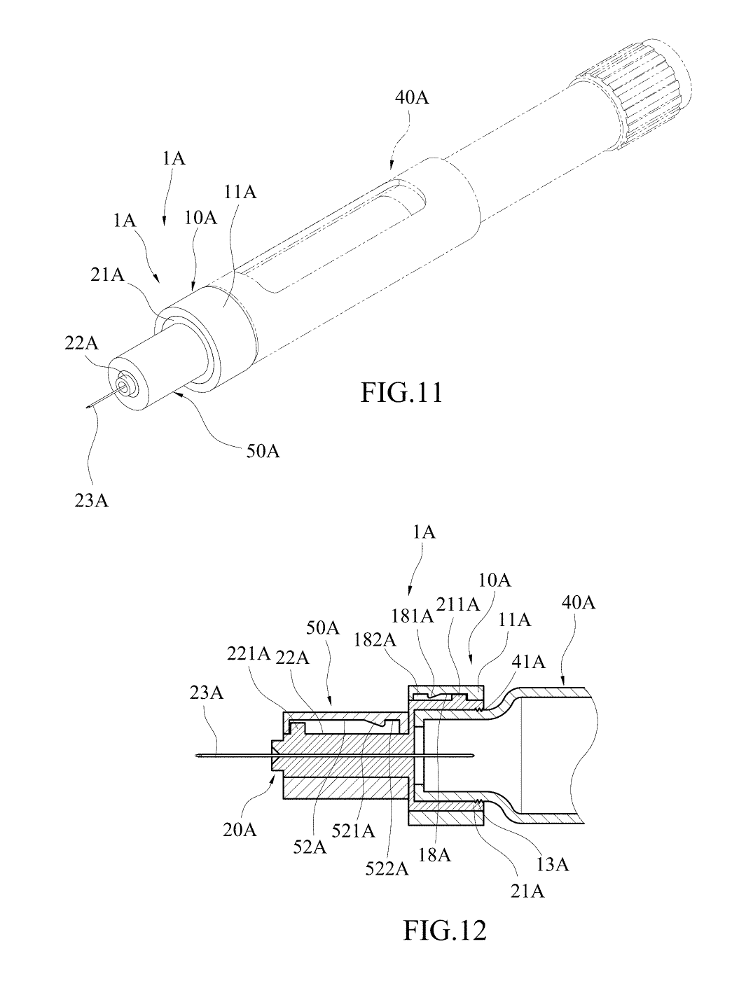

Referring to FIGS. 8 to 14, a safety syringe 1A for injecting regular insulin in accordance with a second preferred embodiment of the invention is shown. The characteristics of the second preferred embodiment are discussed in detail below.

A sleeve assembly 10A, a needle assembly 20A, and a protective sleeve 50A are provided. The sleeve assembly 10A includes a sleeve 11A, an axial channel 12A through the sleeve 11A with the needle assembly 20A mounted therein as detailed later, a rear annular threaded rim 13A on an inner surface of the sleeve 11A, a first groove 17A on the inner surface of the sleeve 11A and open to a front end, a second groove 18A on the inner surface of the sleeve 11A and parallel to the first groove 17A, the second groove 18A having a projection 181A inclined toward a rear end, and a cavity 182A adjacent to the projection 181A and proximate the front end, and a third groove 19A interconnecting a rear end of the first groove 17A and a rear end of the second groove 18A.

The needle assembly 20A includes a hollow cylinder 21A having a protrusion 211A formed on an outer surface, the sleeve 11A placed on the cylinder 21A with the protrusion 211A complimentarily disposed in the first groove 17A, a needle hub 22A formed on a front end of the cylinder 21A and having a protuberance 221A formed on an outer surface, a hypodermic needle 23A disposed through the needle hub 22A, the protective sleeve 50A placed on the needle hub 22A, and a needle protector 24A releasably placed on both a front end of the needle hub 22A and the hypodermic needle 23A and a cap 25A placed on the outer side of the safety syringe 1A prior to use.

The vial 40A includes front external threads 41A configured to threadedly secure to the annular threaded rim 13A to fasten the vial 40A and the sleeve assembly 10A together. The hollow cylindrical protective sleeve 50A includes a lengthwise guide groove 51A formed on an inner surface, the protuberance 221A being disposed in the guide groove 51A after placing the protective sleeve 50A on the needle hub 22A, a lengthwise positioning groove 52A formed on the inner surface and parallel to the guide groove 51A, the positioning groove 52A having a riser 521A inclined toward a forward end, and a well 522A proximate a rear end.

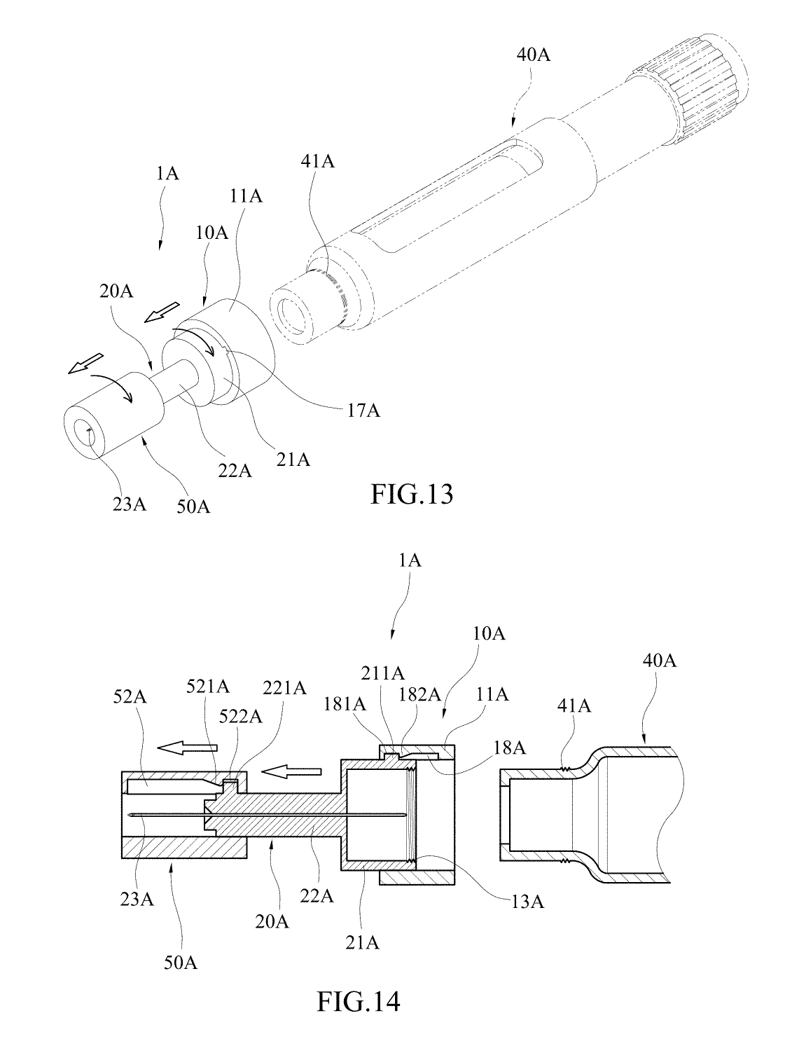

A medical employee may remove the cap 25A and the needle protector 24A prior to injecting liquid medicine (e.g., regular insulin) in the vial 40A into the skin of a patient through the hypodermic needle 23A. After use (see FIGS. 13 and 14), the medical employee may detach the empty vial 40A from the sleeve assembly 10A, rotate the protective sleeve 50A (as indicated by one clockwise arrow), pull the protective sleeve 50A until the protuberance 221A passes the riser 521A to enter the well 522A to be permanently locked therein, rotate the sleeve assembly 10A (as indicated by the other clockwise arrow), pull the sleeve assembly 10A until the protrusion 211A passes the projection 181A to enter the cavity 182A to be permanently locked therein. Thereafter, the medical employee may properly dispose the used safety syringe 1A.

The first, second and third grooves 17A, 18A and 19A may be eliminated from the sleeve 11A, and the protrusion 211A may be eliminated from the needle assembly 20A in the embodiment without departing the spirit and scope of the appended claims.

While the invention has been described in terms of preferred embodiments, those skilled in the art will recognize that the invention can be practiced with modifications within the spirit and scope of the appended claims.

What is claimed is:

1. A safety syringe (1) for injecting regular insulin comprising:
a sleeve assembly (10);
a needle assembly (20); and
a protective covering (30);
wherein:
the sleeve assembly (10) is threadedly secured to a via (40) containing regular insulin and includes an axial channel (12) with the needle assembly (20) placed therein, and two opposite pivot pins (14) formed on an outer surface;
the needle assembly (20) includes a needle hub (22) formed on a front end, and a hypodermic needle (23) disposed through the needle hub (22);
the protective covering (30) includes two opposite arms (31) pivotably secured to the pivot pins (14), a space (32), a slot (33) parallel to the arms (31) and communicating with the space (32), and at least one lock member (34) at an end of the slot (33); and
after injection, a clockwise rotation of the protective covering (30) until the lock member (34) passes through the needle hub (22), and the hypodermic needle (23) passes through the slot (33) to conceal in the space (32).

2. The safety syringe for injecting regular insulin of claim 1, wherein the at least one lock member (34) is inclined.

3. The safety syringe for injecting regular insulin of claim 1, wherein the at least one lock member (34) is inclined toward the space (32).

4. The safety syringe for injecting regular insulin of claim 1, wherein the number of the at least one lock member (34) is two and the lock members (34) are opposite.

5. The safety syringe for injecting regular insulin of claim 1, wherein the number of the at least one lock member (34) is two and the lock members (34) are offset.

6. The safety syringe for injecting regular insulin of claim 1, wherein the sleeve assembly (10) further comprises two sets of upper and lower limit members (15, 16) on an outer surface.

7. The safety syringe for injecting regular insulin of claim 6, wherein the protective covering (30) further comprises a gripping member (35) on a rear surface.

8. The safety syringe for injecting regular insulin of claim 1, further comprising a cap (25) placed on the sleeve (11), the cap (25) including a rear enlargement (251), a forward member (252), two left and right sides spaced cuts (253, 253) and one lower cut on the enlargement (251).

9. A safety syringe (1A) for injecting regular insulin comprising:
a sleeve assembly (10A);
a needle assembly (20A); and
a protective sleeve (50A);
wherein:
the sleeve assembly (10A) is threadedly secured to a via (40A) containing regular insulin and includes an axial channel (12A) with the needle assembly (20A) placed therein;
the needle assembly (20A) includes a hollow cylinder (21A), a needle hub (22A) formed on a front end of the hollow cylinder (21A) and having a protuberance (221A) formed on an outer surface, and a hypodermic needle (23A) disposed through the needle hub (22A);
the protective sleeve (50A) includes a lengthwise guide groove (51A) formed on an inner surface with the protuberance (221A) disposed therein after placing the protective sleeve (50A) on the needle hub (22A), a lengthwise positioning groove (52A) formed on the inner surface and parallel to the guide groove (51A), the positioning groove (52A) having a riser (521A) and a well (522A); and
after injection, a rotation of the protective sleeve (50A) and a pulling of the protective sleeve (50A) until the protuberance (221A) passes the riser (521A) to enter the well (522A) to be permanently locked therein.

10. The safety syringe for injecting regular insulin of claim 9, wherein the riser (521A) is inclined toward a forward end.

11. The safety syringe for injecting regular insulin of claim 9, wherein the sleeve assembly (10A) further comprises a first groove (17A) formed on the inner surface and being open to a front end, a second groove (18A) formed on the inner surface and being parallel to the first groove (17A), the second groove (18A) having a projection (181A) and a cavity (182A) adjacent to the projection (181A), and a third groove (19A) interconnecting a rear end of the first groove (17A) and a rear end of the second groove (18A); the hollow cylinder (21A) includes a protrusion (211A) formed on an outer surface; and the protrusion (211A) is complimentarily disposed in the first groove (17A) in a prior to use position.

12. The safety syringe for injecting regular insulin of claim 9, wherein the projection (181A) is inclined toward a rear end.
